(19) United States
(12) Patent Application Publication
(10) Pub. No.: US 2010/0258129 A1
(43) Pub. Date: Oct. 14, 2010

(54) WATER TRAP FOR A BREATHING TUBE

(75) Inventors: André HUSCHKE, Hamburg (DE); Carina BENTRUP, Lubeck (DE); Marco ZELK, Hamberge (DE); Thomas REINBOTH, Hamburg (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 12/723,817

(22) Filed: Mar. 15, 2010

(30) Foreign Application Priority Data
Apr. 11, 2009 (DE) 10 2009 017 274.2

(51) Int. Cl. A61M 16/00; A62B 7/10

(52) U.S. Cl. 128/205.27

(57) ABSTRACT

A water trap (1) for a breathing tube (2) includes a collection tank (3) for water of condensation and at least one device (4) for attaching the collection tank (3) to the breathing tube (2). The attachment is such that the water of condensation can be drawn off from the breathing tube (2) at any desired point of the breathing tube (2). The water trap (1) has at least one device (5) for preparing at least one opening in the breathing tube (2). The water trap (1) is manufactured at a low cost and makes safer and more reliable handling possible.

### CROSS REFERENCE TO RELATED APPLICATIONS

This application claims the benefit of priority under 35 U.S.C. §119 of German Patent Application DE 10 2009 017 274.2 filed Apr. 11, 2009, the entire contents of which are incorporated herein by reference.

### FIELD OF THE INVENTION

The present invention pertains to a water trap with a collection tank for water of condensation, to a respiration system with a respirator (also known as a ventilator) for the artificial respiration of a patient, at least one breathing tube and with a water trap with a collection tank for water of condensation and to a process for providing a breathing tube with a water trap.

### BACKGROUND OF THE INVENTION

Respirators as medical devices are used for the artificial respiration of patients. The air is sent to the patient for the patient's artificial respiration through a tube system.

Condensation of the humid air may occur in the respiration system during the artificial respiration of the patient with the respirator. Water of condensation is then formed in the breathing tube. The water of condensation leads to an increase in the air resistance in the tube system and may reach the patient's lungs during the patient's artificial respiration. To prevent this, water traps are used in the breathing tubes, which water traps collect the water of condensation formed in the breathing tubes, so that the above-described hazards and risks can no longer occur. The water traps have, in general a removable collection tank, which is to be emptied at regular intervals.

U.S. Pat. No. 4,457,305 shows a water trap of this type for a breathing tube. A connection part, to which the collection tank can be attached, is integrated into the breathing tube. The collection tank is detachably connected to the connection part, so that the collection tank can be removed for its emptying. When the collection tank has been removed, a valve prevents air from being able to flow into the environment from the breathing tube or vice versa when the collection tank is not present at the connection part. However, the water trap can be used at the intended connection part within the breathing tube only. Thus, it is disadvantageously not possible to arrange the water trap at any desired location of the breathing tube, so that the water trap frequently cannot be used at the deepest point of the breathing tube. Optimal and reliable draining of water of condensation from the breathing tube is not guaranteed.

### SUMMARY OF THE INVENTION

The object of the present invention is therefore to make available a water trap for a breathing tube, a respiration system and a process for manufacturing a breathing tube with a water trap or a respiration system, in which the water of condensation can be drawn off from the breathing tube at any desired point of the breathing tube. The water trap and the respiration system shall be able to be manufactured at a low cost and make safer and more reliable handling possible.

This object is accomplished with a water trap for a breathing tube, comprising a collection tank for water of condensation and at least one means for attaching the collection tank to the breathing tube, wherein the water trap has at least one device for preparing at least one opening in the breathing tube. An opening for drawing off the water of condensation from the breathing tube can be prepared with the device on the breathing tube at any desired point. It is thus guaranteed in an advantageous manner that the water of condensation is drawn off at the deepest point (lowest vertical location) of the breathing tube. The user of the water trap can thus build up the breathing tube or the respiration system and subsequently arrange the water trap at the deepest point of the breathing tube after building up the respiration system at the breathing tube.

The device is preferably connected rigidly to the device. The device for preparing at least one opening in the breathing tube may also be a separate component not connected to the rest of the water trap. The device is shipped, for example, as an auxiliary part for the (rest of the) water trap. Such a system or kit with the (rest of the) water trap and with the device for preparing at least one opening in the breathing tube is also considered to be a water trap according to the present invention.

In particular, the at least one opening can be prepared by the at least one device in the breathing tube such that the breathing tube is processed by machining and/or cutting and/or by punching and/or by removing material and/or by piercing.

In another embodiment, the at least one opening is prepared by the at least one device in the breathing tube later, so that the opening can be formed at any desired point of the breathing tube.

In another embodiment, the at least one device has at least one mandrel and/or at least one blade and the mandrel preferably has a duct for passing through the water of condensation. For example, an opening, through which the water of condensation flows out, can be cut into the breathing tube by means of the blade. The mandrel pierces the breathing tube or the wall of the breathing tube and thus prepares an opening in the breathing tube. As a result, the water of condensation can flow out between the mandrel and the wall of the breathing tube. The mandrel preferably has a duct for passing through the water of condensation, so that the water of condensation can be additionally drawn off through this duct or it can be drawn off exclusively from the breathing tube. The duct in the mandrel thus forms an opening in the breathing tube for drawing off the water of condensation.

A positive-locking attachment and/or attachment in substance and/or nonpositive connection can be embodied by means of the at least one means for attaching the collection tank to the breathing tube in a supplementary embodiment. Connection in substance may be established, for example, by means of bonding and/or nonpositive attachment can be embodied, for example, by means of clamping the breathing tube onto the at least one means.

The at least one means preferably comprises a clamp and/or a clip. A clamp or clip is placed around the breathing tube and it will then attach the water trap to the breathing tube.

In one variant, the at least one means comprises two half shells of a U-shaped or V-shaped cross section and the two half shells can be connected to one another by means of at least one fixing means, so that the breathing tube can be attached between the two half shells connected to one another.

The at least one fixing means is preferably a snap-in, bonding, welding and/or locking mechanism.

In another embodiment, the fixing means for connecting the two U-shaped or V-shaped half shells is a clamp and/or clip. The clamp and/or clip is thus placed around the two U-shaped or V-shaped half shells and thus it connects these to one another.

In another embodiment, the external diameter or the outer circumference of the breathing tube is larger than the internal diameter or the inner circumference of the clamp or clip or of the two half shells prior to attachment, so that the breathing tube is clamped within the clamp or clip or two half shells in a nonpositive manner by means of an elastic deformation of the breathing tube during the attachment of the breathing tube.

In particular, the clamp or clip or at least one half shell has ribs or webs on the inside for positive-locking meshing with tangential recesses, especially ring grooves prepared on the outside of the breathing tube.

In another embodiment, the water trap has a sealing element for sealing a fluid-carrying connection from the breathing tube to the collection tank against the environment and/or a process described herein can be carried out.

In another embodiment, the sealing element is at least one rib or at least one web, which is formed on the inside on the at least one half shell and/or the sealing element is an elastic seal and/or an elastic sealing ring, which is preferably arranged on the outside around the breathing tube.

A respiration system according to the present invention, comprising a respirator for the artificial respiration of a patient, at least one breathing tube, e.g., an inspiration tube and an expiration tube, with a water trap and with a mouthpiece, wherein the water trap is designed according to a water trap described in this patent application and/or a process described in this patent application can be carried out.

A process for preparing a breathing tube with a water trap, especially with a water trap described in this patent application, or a respiration system, especially a respiration system described in this patent application, with the steps of making available (providing) a breathing tube, of making available a collection tank for water of condensation, of making available at least one means for attaching the collection tank to the breathing tube, attachment of the collection tank to the breathing tube with the at least one means, preparation of a water-carrying connection from the interior of the breathing tube into the collection tank for carrying water of condensation from the interior of the breathing tube into the collection tank, wherein the water-carrying connection is established by preparing an opening in the breathing tube.

In another variant, the opening is prepared in the breathing tube by machining and/or cutting and/or punching and/or by removing material and/or by piercing.

In another embodiment, the opening is prepared in the breathing tube by means of a blade and/or a mandrel.

In particular, when removing the collection tank from the breathing tube, the fluid-carrying connection present in the breathing tube because of the opening from the breathing tube to the outside of the breathing tube is closed, especially by means of a valve, so that no air will flow from the breathing tube into the environment and/or vice versa.

An exemplary embodiment of the present invention will be described in more detail below with reference to the drawings attached. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is an exploded view of a water trap with a breathing tube;

FIG. 2 is a perspective view of the water trap and of the breathing tube according to FIG. 1, wherein the water trap is attached to the breathing tube;

FIG. 3 is a side view of the water trap and of the breathing tube according to FIG. 1, wherein the water trap is attached to the breathing tube;

FIG. 4 is a section A-A according to FIG. 3 of the water trap and of the breathing tube;

FIG. 5 is a section B-B according to FIG. 4 of the water trap and of the breathing tube;

FIG. 6 is a perspective view of a first half shell of the water trap with a mandrel;

FIG. 7 is a side view of the first half shell of the water trap with the mandrel according to FIG. 6;

FIG. 8 is a perspective view of a second half shell of the water trap with a connecting branch for a collection tank;

FIG. 9 is a side view of the second half shell of the water trap with the connecting branch according to FIG. 8; and

FIG. 10 is a schematic view showing a respiration system according to the invention with a respirator, one or more breathing tubes, a mouthpiece and the water trap according to the invention.

### DESCRIPTION OF THE PREFERRED EMBODIMENTS

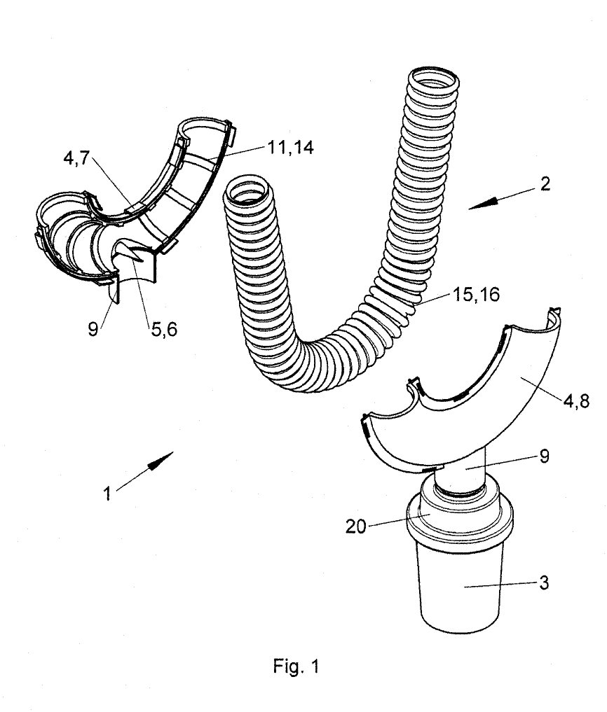

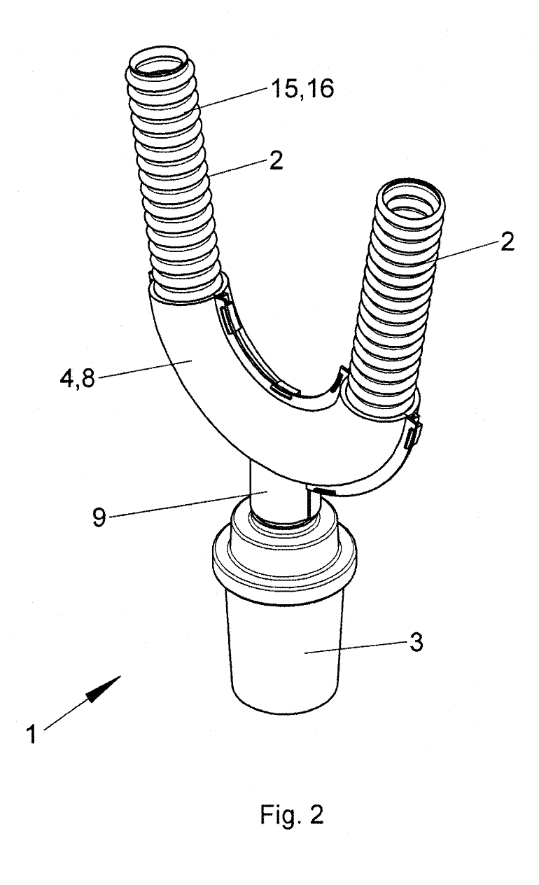

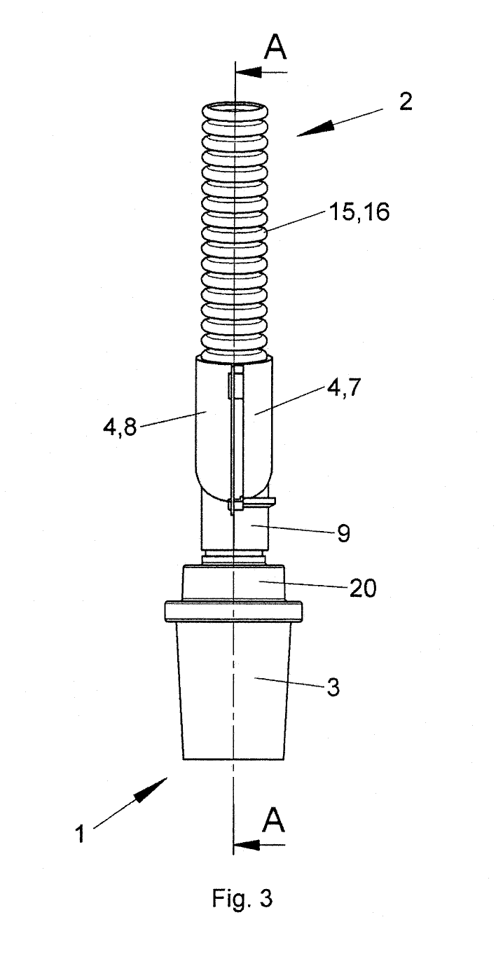

Referring to the drawings in particular, a water trap 1 shown in FIGS. 1 through 5 is used to draw off water of condensation from a breathing tube 2 of a respiration system. Water trap 1 thus prevents larger quantities of water of condensation from collecting in the breathing tube 2, as a result of which the air resistance in the breathing tube 2 could rise or water of condensation could enter the lungs of the patient to be respirated.

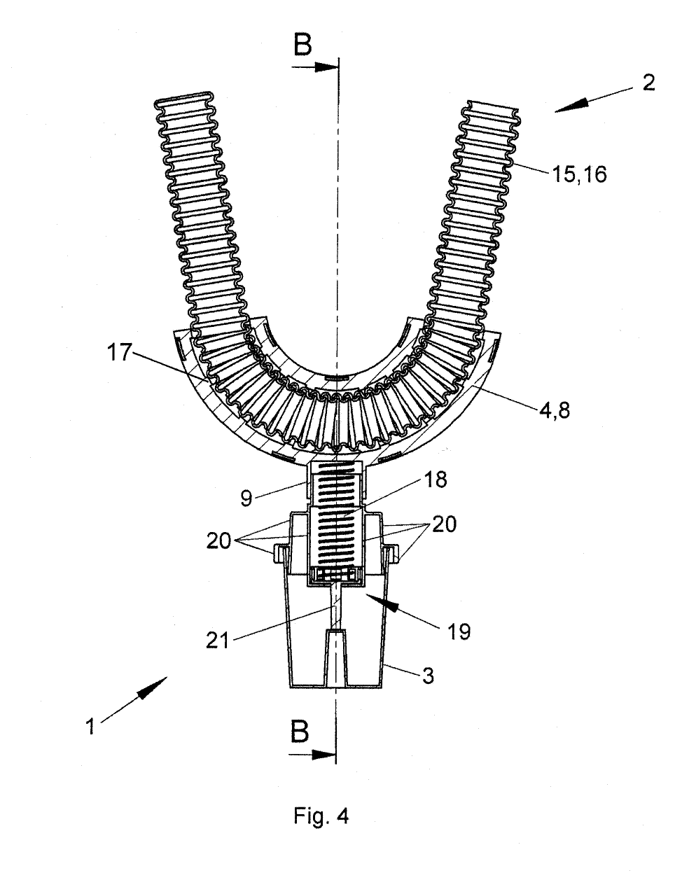

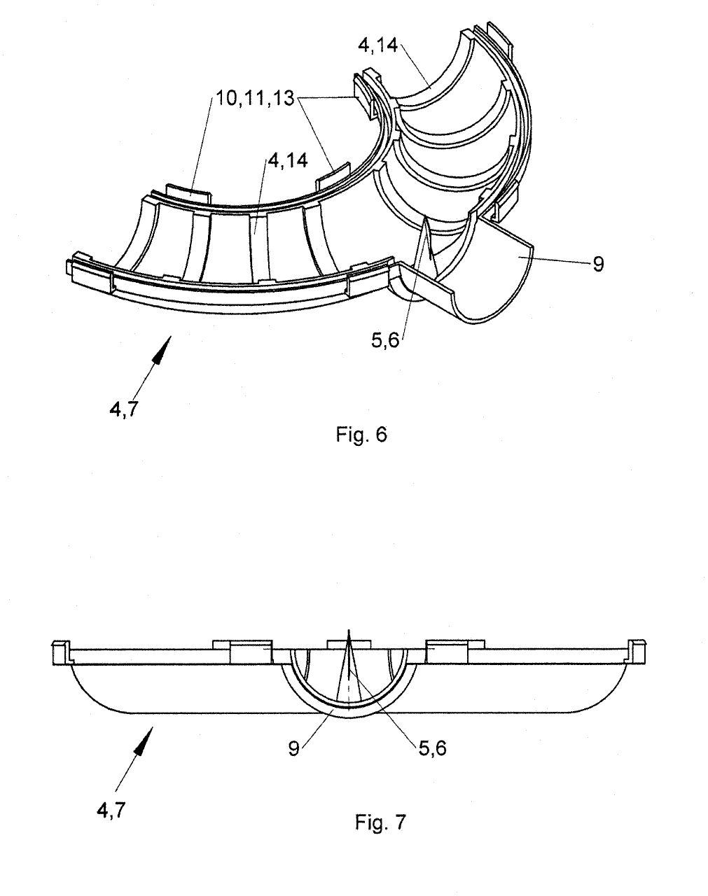

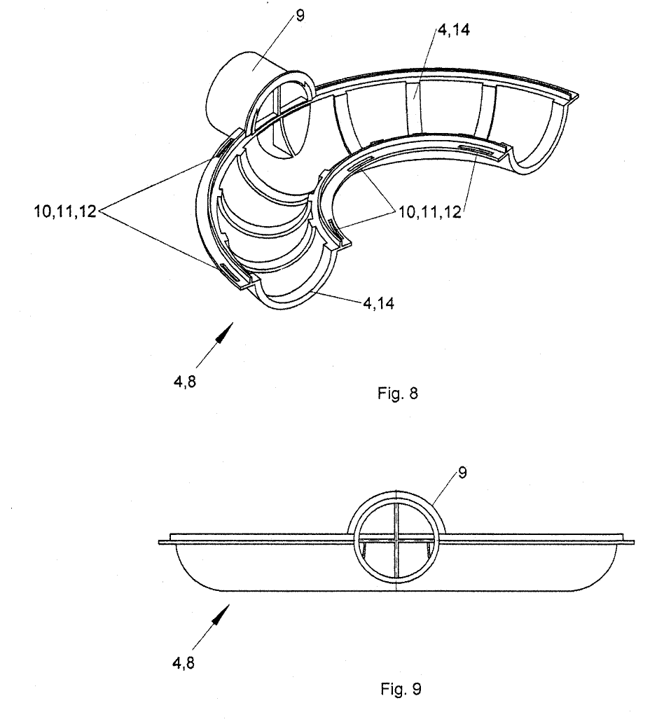

Water trap 1 comprises a collection tank 3 for collecting water of condensation and means 4 for attaching the collection tank 3 to the breathing tube 2. The means 4 comprises a first half shell 7 and a second half shell 8 (FIGS. 1, 2, 3 and 5). A device 5 for preparing an opening in the breathing tube 2, which is designed as a mandrel 6 (FIGS. 1, 6 and 7), is present at the first half shell 7. The flexible breathing tube 2 consisting of a plastic has tangential recesses 15 as ring grooves 16 (FIGS. 1 through 5). The first and second half shells 7, 8 have a U-shaped or V-shaped cross section and are curved in their longitudinal extension, so that the breathing tube 2 is curved with the breathing tube 2 inserted into the first and/or second half shell 7, 8 (FIG. 4). Half of a connecting branch 9 is formed on the first and second half shell 7, 8. Contrary to this, the connecting branch 9 may also be formed entirely on the second half shell 8 without the mandrel 6 (shown in FIG. 8 only). Ribs 14 are present on the inside on the first and second half shells 7, 8 (FIGS. 6 and 8). Ribs 14 mesh with the ring grooves 16 of the breathing tube 2 when the first and second half shells 7, 8 are arranged on the breathing tube 2, so that positive-locking attachment of the breathing tube 2 to the first and second half shells 7, 8 is possible as a result. In addition, the internal diameter of the first and second half shells 7, 8 when the first and first half shells 7, 8 are connected to one another and no breathing tube 2 is present between the first and second half shells 7, 8 may be sized to provide a non-positive attachment. In this case, the internal diameter within the first and second half shells 7, 8 is smaller than the external diameter of the breathing tube 2. Thus, elastic deformation and a reduction of the size of the breathing tube 2 between the first and second half shells 7, 8 take place when the breathing tube 2 is attached between the first and second half shells 7, 8, so that an additional (or in the alternative only a) nonpositive connection is established hereby between the first and second half shells 7, 8 and the breathing tube 2. Ribs 14 act, in addition, as sealing elements 17 for sealing a fluid-carrying connection from the interior of the breathing tube 2 into the collection tank 3 against the environment.

The first and second half shells 7, 8 are connected to one another by means of a fixing means 10 designed as a locking mechanism 11, so that the breathing tube 2 is clamped as a result between the first and second half shells 7, 8. The first half shell 7 has detents 13 for this and the second half shell 8 has locking recesses 12 (FIGS. 6 and 8). When the first half shell 7 and the second half shell 8 are brought together, detents 13 are introduced into the second half shell 8 and are locked there, so that a positive-locking and nonpositive connection is established hereby between the first half shell 7 and the second half shell 8.

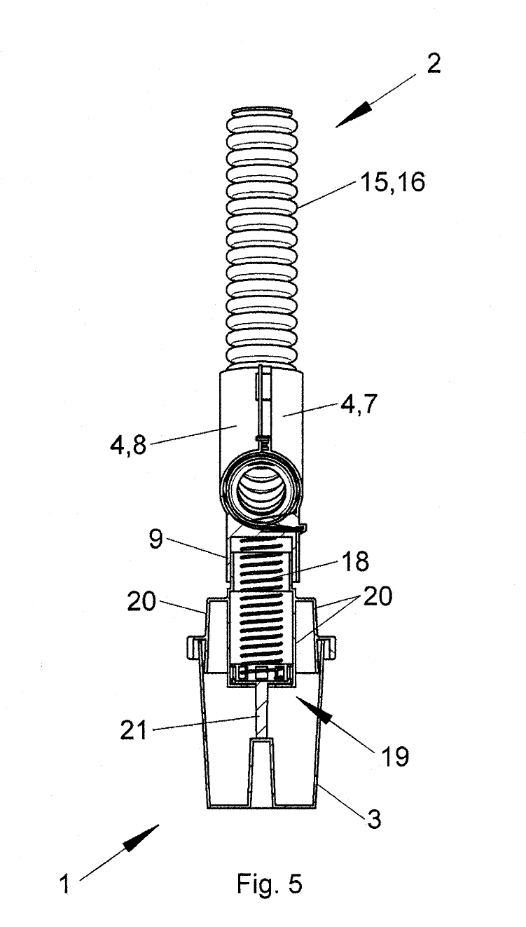

When the first half shell 7 and the second half shell 8 are arranged on the breathing tube 2, mandrel 6 pierces the breathing tube 2 and thus prepares an opening in the wall of the breathing tube 2. After the first and second half shells 7, 8 have been attached and the wall of the breathing tube 2 has been pierced with mandrel 6, a connection element 20 (FIGS. 1 through 5) is attached to the connecting branch 9. The collection tank 3 is then attached detachably to connection element 20. The connection between connecting branch 9 and connection element 20 as well as between connection element 20 and collection tank 3 is preferably a detachable connection, e.g., a locking or screw type connection. A spring 18 and a valve 19 are present within connection element 20. In addition, a lifting bolt 21 is formed concentrically within collection tank 3 (FIGS. 4 and 5). Furthermore, the connection between connecting branch 9 and connection element 20 as well as between connection element 20 and collection tank 3 is sealed in a fluid-tight manner, so that no air can be discharged into the environment from breathing tube 2 through the opening prepared by means of mandrel 6 and/or vice versa.

The water of condensation collecting in the breathing tube 2 thus flows through the opening prepared in the breathing tube 2 by means of mandrel 6, then through connection element 20 and then into collection tank 3, in which the water of condensation collects. The detachable connection between collection tank 3 and connection element 20 is to be separated to empty collection tank 3, so that collection tank 3 can then be emptied. After removal of collection tank 3 from connection element 20, valve 19, to which pressure is applied by spring 18, closes the fluid-carrying connection from connection element 20 to collection tank 3. No air can thus flow out of the breathing tube 2 into the environment and/or vice versa when collection tank 3 has been removed. The emptying of collection tank 3 can thus also be carried out advantageously during the respiration of a patient.

When collection tank 2 is reattached to connection element 20, valve 19 is lifted by the lifting bolt 21, so that water of condensation can again flow from the breathing tube 2 into the collection tank because valve 19 has been opened by the lifting bolt 21 against the force of spring 18.

The half shells 7, 8 consisting of plastic are manufactured such that a fluid-tight connection becomes established at the connection points between the two half shells 7, 8. Furthermore, the elastic breathing tube 2 is clamped in between the two half shells 7, 8, so that no air can flow out here from the breathing tube into the environment. In addition, ribs 14 act as additional sealing elements 17 for sealing the breathing tube 2 against the environment at the two half shells 7, 8.

The first half shell 7 with the mandrel 6 as well as the second half shell 8 without mandrel 6 are each made of plastic in one piece with or without connecting branch 9. This also applies to the collection tank 3, the connection element 20 and the lifting bolt 21, which is made in one piece with collection tank 3. The modulus of elasticity or the hardness of the two half shells 7, 8 may be identical or different. For example, the first half shell 7 may be hard and the second half shell 8 soft or vice versa or both half shells 7, 8 are hard or both half shells 7, 8 are soft. In case of a hard design of the two half shells 7, 8, it is possibly necessary to provide an elastic seal (not shown) at the junction between the two half shells 7, 8 in order to guarantee sealing of the breathing tube 2 with the opening against the environment. Furthermore, the modulus of elasticity or the hardness of the two half shells 7, 8 and of the breathing tube 2 may be equal or different. The modulus of elasticity or hardness of the breathing tube 2 may be greater than the modulus of elasticity or hardness of the two half shells 7, 8 or vice versa. For example, the breathing tube 2 is soft and the two half shells 7, 8 are hard. The modulus of elasticity or hardness of the breathing tube 2 and of the two half shells 7, 8 may also be equal. It may be necessary in case of a hard or only poorly deformable design of both the breathing tube 2 and of the two half shells 7, 8 to form the ring grooves 16 as elastic seals.

On the whole, considerable advantages are associated with the water trap 1 according to the present invention for the breathing tube 2. Water trap 1 may be arranged at any desired point on the breathing tube 2 because the opening for drawing off water of condensation from breathing tube 2 can be prepared later on the breathing tube 2 at any desired point by means of device 5. As a result, the water trap 1 can be arranged at the deepest point of the breathing tube 2 in an especially advantageous manner, so that reliable and optimal drainage of the water of condensation from the breathing tube 2 is guaranteed as a result.

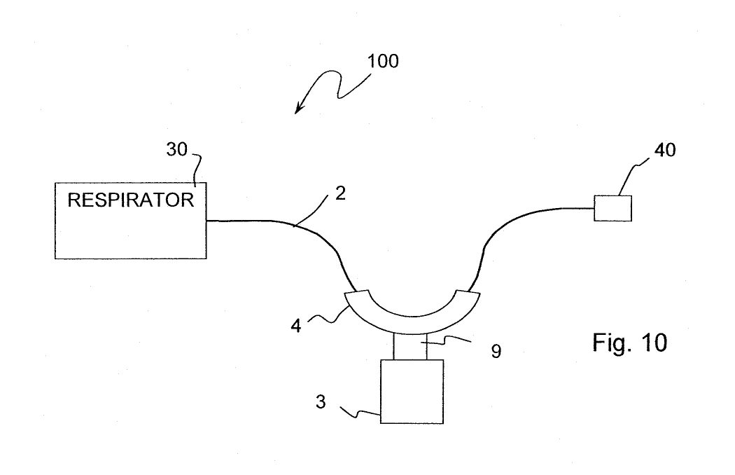

FIG. 10 shows a respiration system according to the invention. The respiration system includes at least a respirator 30. One or more breathing tubes 2 is connected to the respirator 30. The breathing tube 2 leads to a mouthpiece 40. As described above, the breathing tube 2 is connected to a water trap 1 as described above. In particular the water trap has an attaching means 4 for attaching the collection tank 3 to the breathing tube 2. Additionally, the water trap is provided with an opening preparation device for preparing at least one opening in the breathing tube.

While specific embodiments of the invention have been described in detail to illustrate the application of the principles of the invention, it will be understood that the invention may be embodied otherwise without departing from such principles.

### LIST OF REFERENCE NUMBERS

1 Water trap
2 Breathing tube
3 Collection tank for water of condensation
4 Means for attaching the collection tank
5 Device for preparing an opening
6 Mandrel
7 First half shell with mandrel
8 Second half shell without mandrel
9 Connecting branch
10 Fixing means
11 Locking mechanism
12 Locking recess
13 Detent
14 Rib
15 Tangential recess
16 Ring groove
17 Sealing element
18 Spring
19 Valve
20 Connection element
21 Lifting bolt

What is claimed is:

1. A water trap for a breathing tube, the water trap comprising:
a collection tank for collection of water of condensation;
an attaching means for attaching said collection tank to the breathing tube; and
an opening preparation device for preparing at least one opening in the breathing tube.

2. A water trap in accordance with claim 1, wherein said opening preparation device prepares an opening in the breathing tube by one or more of machining and/or cutting and/or punching and/or by removing material and/or by piercing.

3. A water trap in accordance with claim 1, wherein said opening preparation device comprises at least one of a mandrel and blade and mandrel with a duct for passing through the water of condensation.

4. A water trap in accordance with claim 1, wherein said attaching means provides one of a positive-locking attachment, attachment in substance and a nonpositive attachment of said collection tank to said breathing tube.

5. A water trap in accordance with claim 4, wherein said attaching means comprises a clamp and/or clip.

6. A water trap in accordance with claim 5, wherein said attaching means comprises:
two half shells of a U-shaped or V-shaped cross section; and
a fixing means connecting said two half shells to one another, the breathing tube being attached between the two half shells with the two half shells connected to one another.

7. A water trap in accordance with claim 6, wherein said fixing means comprises a snap-in and/or locking mechanism.

8. A water trap in accordance with claim 6, wherein an external diameter or outer circumference of the breathing tube is larger than an internal diameter or inner circumference of the clamp or clip or two half shells prior to the attachment, so that the breathing tube is clamped within the clamp or clip or said two half shells in a nonpositive manner by means of an elastic deformation of the breathing tube during the attachment of the breathing tube.

9. A water trap in accordance with claim 6, wherein the clamp or clip or at least one half shell has ribs or webs on an inner surface thereof for positive-locking engagement with tangential recesses located on an outer surface of the breathing tube.

10. A water trap in accordance with claim 1, wherein the water trap has a sealing element for sealing a fluid-carrying connection from said breathing tube to said collection tank against the environment.

11. A respiration system comprising:
a respirator for artificial respiration of a patient;
at least one breathing tube for at least one of inspiration and expiration;
a mouthpiece; and
a water trap comprising a collection tank for collection of water of condensation, an attaching means for attaching said collection tank to the breathing tube and an opening preparation device for preparing at least one opening in the breathing tube.

12. A water trap in accordance with claim 11, wherein said opening preparation device prepares an opening in the breathing tube by one or more of machining and/or cutting and/or punching and/or by removing material and/or by piercing.

13. A respiration system in accordance with claim 11, wherein said opening preparation device comprises at least one of a mandrel and blade and mandrel with a duct for passing through the water of condensation.

14. A respiration system in accordance with claim 11, wherein said attaching means provides one of a positive-locking attachment, attachment in substance and a nonpositive attachment of said collection tank to said breathing tube.

15. A respiration system in accordance with claim 14, wherein said attaching means comprises a clamp and/or clip.

16. A respiration system in accordance with claim 15, wherein said attaching means comprises:
two half shells of a U-shaped or V-shaped cross section; and
a fixing means connecting said two half shells to one another, the breathing tube being attached between the two half shells with the two half shells connected to one another.

17. A respiration system in accordance with claim 16, wherein an external diameter or outer circumference of the breathing tube is larger than an internal diameter or inner circumference of the clamp or clip or two half shells prior to the attachment, so that the breathing tube is clamped within the clamp or clip or said two half shells in a nonpositive manner by means of an elastic deformation of the breathing tube during the attachment of breathing tube.

18. A respiration system in accordance with claim 16, wherein the clamp or clip or at least one half shell has ribs or webs on an inner surface thereof for positive-locking engagement with tangential recesses located on an outer surface of the breathing tube.

19. A respiration system in accordance with claim 11, wherein the water trap has a sealing element for sealing a fluid-carrying connection from said breathing tube to said collection tank against the environment.

20. A process for providing a breathing tube with a water trap, the process comprising the steps of:
providing a breathing tube;
providing a collection tank for water of condensation;
providing an attachment means for attaching the collection tank to the breathing tube;
attaching the collection tank to the breathing tube with said attaching means;
preparing an opening in breathing tube and establishing a water-carrying connection from an interior of said breathing tube into the collection tank for sending water of condensation from the interior of breathing tube into said collection tank.

21. A process in accordance with claim 20, wherein the opening is prepared in the breathing tube by machining and/or cutting and/or punching and/or by removing material and/or piercing.

22. A process in accordance with claim 20, wherein the opening is prepared in the breathing tube by a blade and/or a mandrel.

23. A process in accordance with claim 20, further comprising a seal structure with a valve for sealing the opening in the breathing tube to prevent fluid flow from inside the breathing tube to the outside of the breathing tube when the collection tank is removed from the breathing tube, whereby no air flows from the breathing tube into the environment and/or vice versa.
